(19) United States
(12) Patent Application Publication
(10) Pub. No.: US 2009/0112215 A1
(43) Pub. Date: Apr. 30, 2009

(54) OPTO-ELECTRIC INDICATORS FOR ORTHOPAEDIC INSTRUMENTS

(76) Inventor: JASON T. SHERMAN, Warsaw, IN (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/926,414

(22) Filed: Oct. 29, 2007

(51) Int. Cl. A61B 17/58; A61B 17/00; A61B 18/18

(52) U.S. Cl. 606/99; 606/45; 606/80

(57) ABSTRACT

An instrument for use in implanting a joint prosthesis into a bone. The instrument includes a rod having a proximal end and a distal end, the proximal end includes a visual indicator. The instrument also includes a housing, such that the rod is moveable in a longitudinal direction relative to the housing. The visual indicator is capable of emitting light when the instrument is in use. As the rod moves in the longitudinal direction, the visual indicator enters into the housing, causing the light emitted to disappear.

### TECHNICAL FIELD OF THE INVENTION

The present invention relates generally to the field of orthopedics, and more particularly, to an implant for use in arthroplasty.

### BACKGROUND OF THE INVENTION

Patients who suffer from the pain and immobility caused by osteoarthritis and rheumatoid arthritis have an option of joint replacement surgery. Joint replacement surgery is quite common and enables many individuals to function properly when it would not be otherwise possible to do so. Artificial joints are usually comprised of metal, ceramic and/or plastic components that are fixed to existing bone.

Such joint replacement surgery is otherwise known as joint arthroplasty. Joint arthroplasty is a well-known surgical procedure by which a diseased and/or damaged joint is replaced with a prosthetic joint. In a typical total joint arthroplasty, the ends or distal portions of the bones adjacent to the joint are resected or a portion of the distal part of the bone is removed and the artificial joint is secured thereto.

There are known to exist many designs and methods for manufacturing implantable articles, such as bone prostheses. Such bone prostheses include components of artificial joints such as elbows, hips, knees and shoulders.

In order to implant the prosthesis, many different instruments may be used. In some instances an instrument, such as a reamer, may include visual markings to aid in the preparation of the bone or insertion of the implant. For example, a reamer may be used to cut holes in the bone or a channel into a long bone. When using a reamer, it is important to only remove the necessary amount of bone. Therefore, many reamers currently in use are often provided with markings to indicate the depth that the bone has been reamed. However, in many existing reamers, the markings are etched onto the metal side and can be difficult for the operator to see while using the reamer.

In other instruments, mechanicals stops are sometimes used to solve this problem. When the reamer gets to the appropriate depth, the stop would effectively prevent the reamer from reaming any further. However, mechanical stops can be difficult and costly to machine. Also, if the stops are adjustable, the stops can sometimes be cumbersome and confusing to use.

Therefore, there is a need for an improved visual indicator on instruments for indicating when an appropriate depth has been achieved.

The present invention is directed to alleviate at least some of the problems with the prior art.

### SUMMARY OF THE INVENTION

According to one embodiment of the present invention, an instrument for use in implanting a joint prosthesis into a bone is provided. The instrument includes a rod having a proximal end and a distal end. The proximal end includes a visual indicator. The instrument also includes a housing. The rod is moveable in a longitudinal direction relative to the housing. The visual indicator is capable of emitting light when the instrument is in use. As the rod moves in the longitudinal direction, the visual indicator enters into the housing, causing the light emitted to disappear.

According to another embodiment of the present invention, a method for providing a visual indicator on an instrument for use in implanting a joint prosthesis into a bone is provided. The method includes providing the instrument. The instrument includes a housing and a rod having a visual indicator. The rod is slidably coupled with the housing. The method also includes using the instrument such that the visual indicator emits a light and sliding the rod into the housing until the visual indicator is in the housing. The light emitted by the visual indicator disappears from view.

According to yet another embodiment of the present invention, an instrument for use in implanting a joint prosthesis into a bone is provided. The instrument includes a rod having a proximal end and a distal end. The proximal end includes a visual indicator. The instrument also includes a housing, such that the rod is moveable in a longitudinal direction relative to the housing. The visual indicator emits light when the instrument is in use. As the rod moves in the longitudinal direction, the visual indicator enters into the housing, causing the visual indicator to cease emitting light.

### BRIEF DESCRIPTION OF THE DRAWINGS

For a more complete understanding of the present invention and the advantages thereof, reference is now made to the following description taken in connection with the accompanying drawings, in which:

FIG. 1 is a plan view of a reamer in position in a long bone and having a visual indicator according to one embodiment of the present invention;

FIG. 2 is a plan view of the reamer of FIG. 1 with the visual indicator in the housing;

FIG. 3 is a plan view of a reamer in position in a long bone and having a plurality of visual indicators according to one embodiment of the present invention;

FIG. 4 is a circuit diagram of the visual indicator according to one embodiment of the present invention;

FIG. 5 is a plan view of a reamer in position in a long bone and having a visual indicator according to one embodiment of the present invention;

FIG. 6 is a plan view of a reamer in position in a long bone and having a plurality of visual indicators according to one embodiment of the present invention; and

FIG. 7 is a flow chart describing the operation of the reamer of FIG. 1 according to one embodiment of the present invention.

### DETAILED DESCRIPTION OF THE INVENTION

Embodiments of the present invention and the advantages thereof are best understood by referring to the following descriptions and drawings, wherein like numerals are used for like and corresponding parts of the drawings.

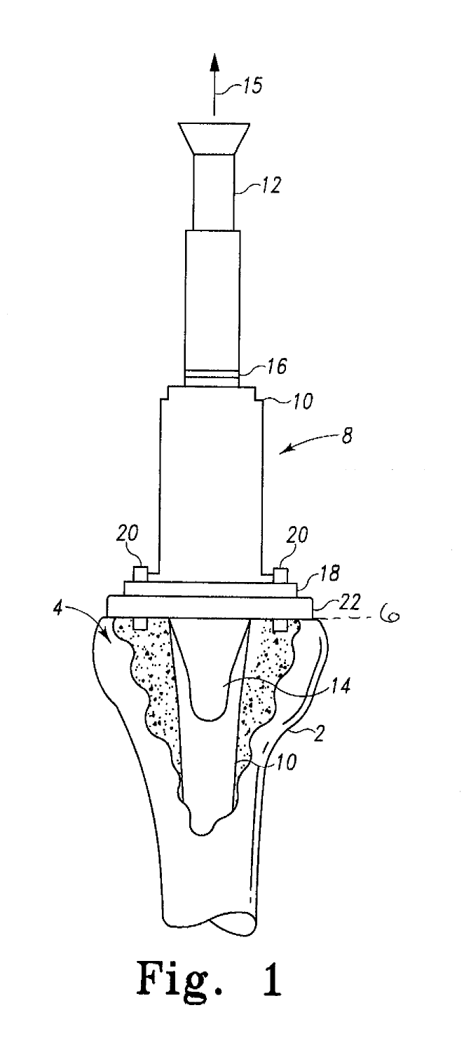

Referring now to FIG. 1 a long bone 2, such as a femur or tibia, is shown. The long bone 2 includes an intermedullary canal 4 into which a prosthesis may be inserted. The long bone 2 is resected along resection line 6 by, for example, a power tool, for example, a saw. The resecting of the long bone 2 exposes the intermedullary canal 4 of the long bone 2. A reamer 8 according to one embodiment of the present invention is positioned in the intermedullary canal 4 of the long bone 2 to form cavity 10 for receiving an orthopedic joint implant. The reamer 8 is used to remove bone and other biological matter from the intermedullary canal 4 to form the cavity 10. The reamer 8 is rotated by use of a connector (not shown) positioned on the reamer 8. The connector may be any standard connector for example a Hudson or an A-O connector. The connector is used to connect to a power tool (not shown) for rotating the reamer 8. The power tool (not shown) may be any standard power tool. It should be appreciated that the reamer 8 may be rotated through the use of the connector by a hand tool for example a “T” shaped handle.

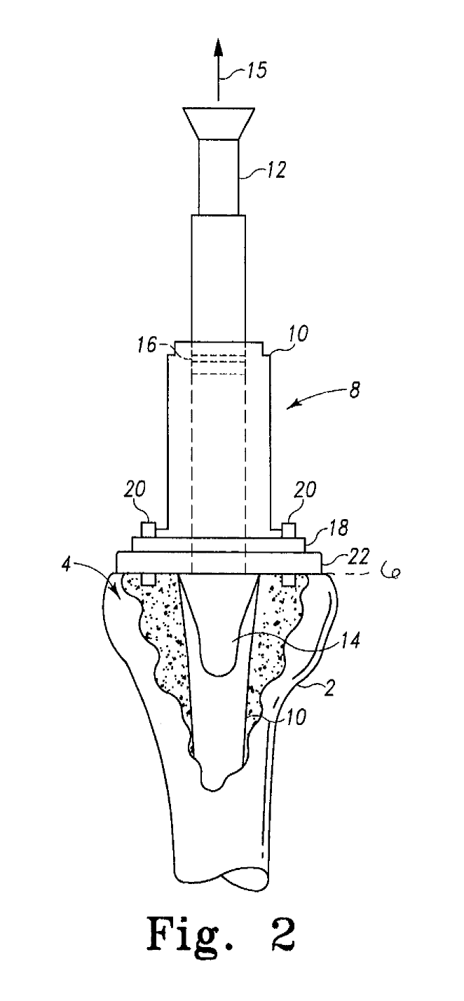

As shown in FIG. 1, the reamer 8 includes a housing 11 and a rod 12 that extends through the housing 11 and includes the cutting edges 14 of the reamer 8. The rod 12 is moveable along a longitudinal direction 15 relative to the long bone 2 and the housing 11. The rod 12 also includes a visual indicator 16. The visual indicator 16 may be an LED or other light source that is capable of shining a beam of light above the housing 11. The light from the visual indicator 16 will provide the user with the ability to know when to stop reaming. Once the light is no longer seen, the user can stop the reamer 8. The operation of the visual indicator will be discussed more below in reference to FIG. 2.

The housing 11 of the reamer 8 includes a base 18. The base 18 includes a plurality of pins 20 that couple the base 18 to a plate 22 and the bone 2. The pins 20, base 18, and plate 22 ensure that the reamer 8 is securely fixed to the bone 2 and that there is not any relative movement between the housing 11 and the bone 2, to ensure a more accurate reaming of the bone.

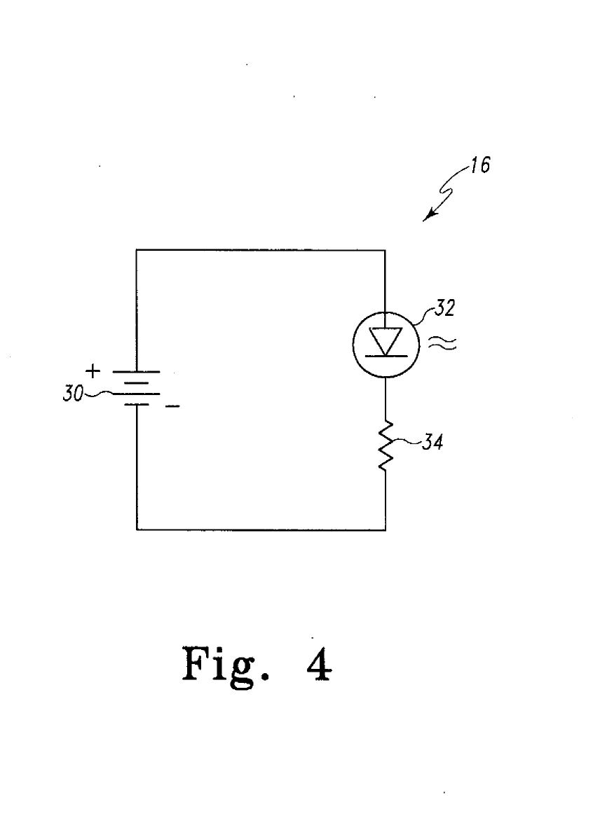

Referring now to both FIGS. 1 and 2, the operation of the visual indicator 16 will be described. Prior to the reamer 8 beginning reaming, the visual indicator 16 will be above the housing 11. In one embodiment, the visual indicator 16 will include a switch that will be activated by the rotation of the rod 8 about the longitudinal axis 15. The switch (not shown) may be a button that is activated by the user. Alternatively, the switch may be a centrifugal switch that is actuated by the rotating of the reamer 8. For example, the centrifugal switch may include a spring that extends outwardly when the reamer 8 begins to spin, completing the circuit (shown without a switch in FIG. 4). Once the reamer 8 stops spinning, the spring would retract, breaking the contact, thereby turning off the light.

As the cutting edges 14 move downward into the long bone 2, the visual indicator 16 also moves downward, eventually moving below the housing 11 as shown in phantom in FIG. 2. Once the visual indicator 16 is below the housing 11, the light it emits is no longer visible. Alternatively, the act of entering the housing may turn the visual indicator 16 off, so that no more light is emitted. Regardless of the method, the user would not see the light any more and would know to stop reaming.

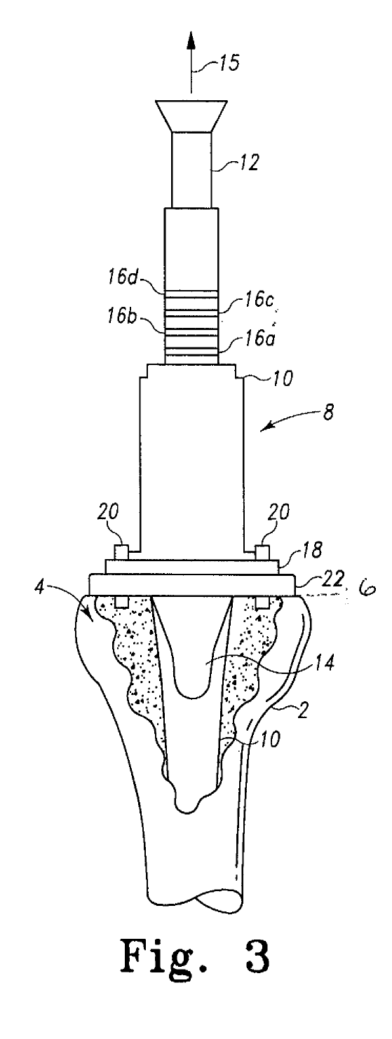

As shown in FIG. 3, some embodiments may include multiple visual indicators 16a, 16b, 16c, 16d. Each visual indicator 16a, 16b, 16c, 16d will emit a different colored light. The spacing of the various visual indicators 16a, 16b, 16c, 16d will indicate different reaming depths. The user (such as the surgeon) will first determine how far the bone 2 needs to be reamed and which color light corresponds to that depth. Once the user begins reaming, all of the visual indicators 16a, 16b, 16c, 16d will emit light. Then, as the rod 12 moves downward, the visual indicators 16a, 16b, 16c, 16d will, one-by-one, descend below the housing 11. Correspondingly, the lights from the visual indicators 16a, 16b, 16c, 16d will, one-by-one, disappear. Once the colored light that corresponds to the depth the user wants to ream can no longer be seen, the user knows to cease reaming.

Turning now to FIG. 4, a circuit diagram of the visual indicator 16 according to one embodiment of the present invention is illustrated. In this embodiment, the visual indicator 16 is a battery-powered LED. As shown, the visual indicator includes a battery 30 connected in series to an LED 32 and to a resistor 34. The positive terminal of the battery 30 is connected to the anode of the LED 32. The cathode of the LED 32 is then connected to the resistor 34, which is in turn connected to the negative terminal of the battery 30. The formula to use to calculate the correct resistor 34 to use is:

R=(Vₚ−Vₗ)Iₗ

where:

Vₚ is the voltage of the power supply (such as a 9 volt battery)

Vₗ is the voltage drop across the LED

Iₗ is the manufacturer rating of the LED

Generally, the voltage drop depends on the color and the brightness of the LED, as shown in the table below.

TABLE 1

| | Typical voltage |
|---|---|
| Diode type | drop (volts) |
| non-high-brightness red | 1.7 volts |
| high-brightness, high-efficiency and low-current red | 1.9 volts |
| orange and yellow | 2 volts |
| green | 2.1 volts |
| bright white, bright non-yellowish green, and most blue | 3.4 volts |
| bright blue and some specialized LEDs | 4.6 volts |

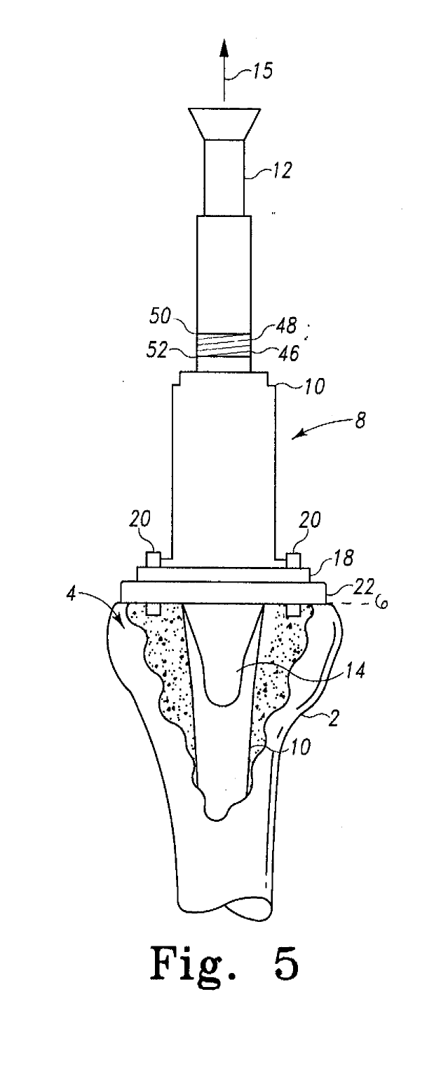

Turning now to FIG. 5, another embodiment of the present invention will be illustrated. As in FIGS. 1-3, the reamer 8 includes a housing 11 and a rod 12. The rod 12 includes a visual indicator 46. In this embodiment, the visual indicator 46 is a light collecting mechanism, such as a long strand of scintillating ambient light collecting fiber optic filament 48, wrapped around the circumference of the rod 12. The rod 12 includes two lips or ridges 50, 52 for keeping the filament 48 in place. The lips 50, 52 may also include outwardly extending protrusions to hold the filament 48 against the rod 12.

The filament 48 collects ambient light over a large surface area and emits an intensified beam of light at the end of the filament 48, as known in the art. Similar to the embodiment described in reference to FIGS. 1 and 2, as the rod 12 (and the visual indicator 46) is lowered into the housing 11, the filament 48 will stop emitting light (or the light will no longer be able to be seen by the user). Because the user will not be able to see the light, the user will know to stop reaming.

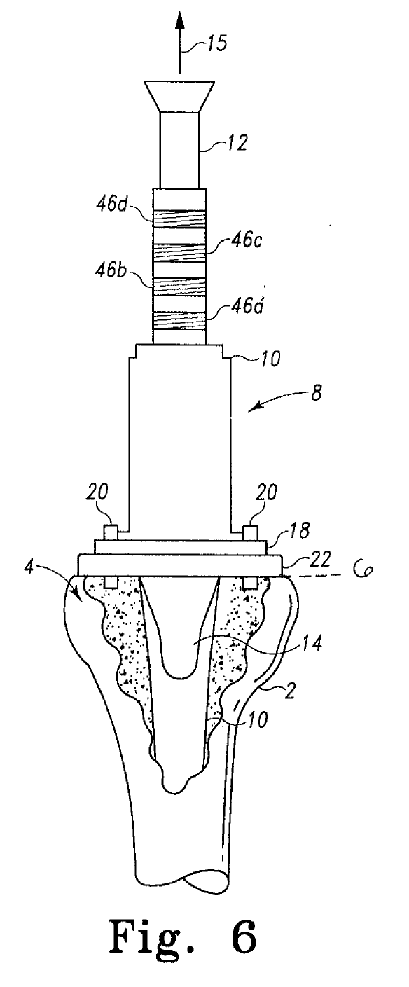

As shown in FIG. 6, the reamer 8 may also include a plurality of light indicators 46a, 46b, 46c, 46d, with each indicator emitting a different color light and corresponding to a different reamer depth.

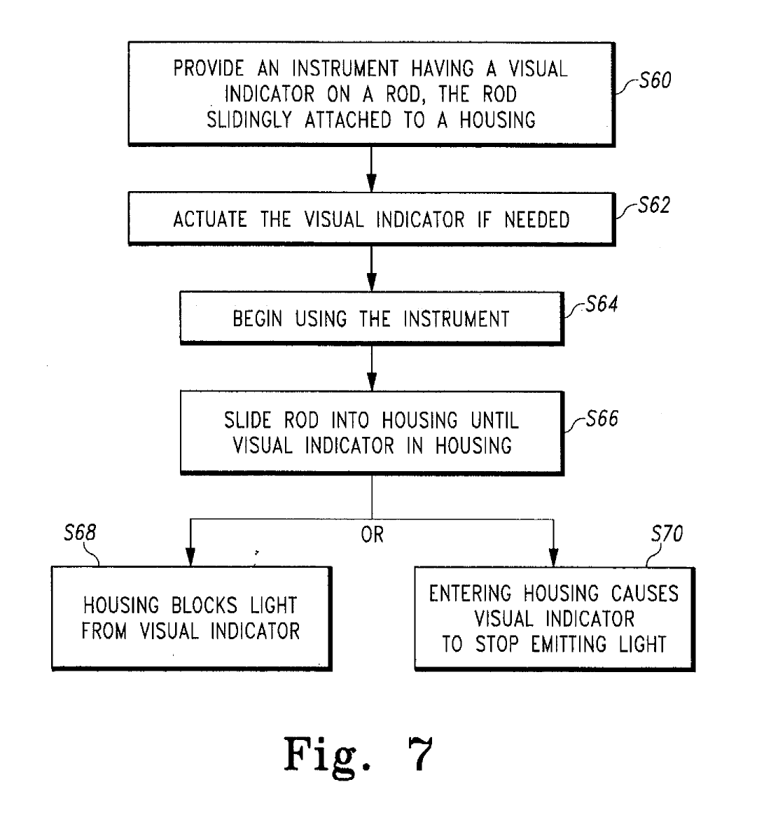

Turning now to FIG. 7, a flow chart illustrating the operation of one embodiment of the present invention is shown. An instrument including a visual indicator on a rod that is slidingly coupled to a housing is provided at step s60. In some embodiments, the user may need to activate the visual indicator at step s62. In other embodiments, such as with the light collecting fiber, the user may not need to activate the visual indicator, and the method may proceed to step s64, in which the user begins to use the instrument. The rod is slid into the housing at step s66 until the visual indicator is within the housing. Then, either the housing blocks the light that is emitting from the visual indicator (step s68) or the act of entering the housing causes the visual indicator to stop emitting light (s70).

Although the embodiments described above were reamers, it should be understood that other types of instruments may also use the visual indicators as described.

Although the present invention and its advantages have been described in detail, it should be understood that various changes, substitutions, and alterations can be made therein without departing from the spirit and scope of the present invention as defined by the appended claims.

1. An instrument for use in implanting a joint prosthesis into a bone, the instrument comprising:
a rod having a proximal end and a distal end, the proximal end including a visual indicator; and
a housing, the rod moveable in a longitudinal direction relative to the housing, the visual indicator capable of emitting light when the instrument is in use, wherein as the rod moves in the longitudinal direction, the visual indicator enters into the housing, causing the light emitted to disappear.

2. The instrument of claim 1, wherein the rod includes a plurality of visual indicators, each of the visual indicators emitting a different color of light.

3. The instrument of claim 2, wherein each of the plurality of visual indicators are separated by a predetermined distance, such that as the rod is moved in the longitudinal direction, the plurality of visual indicators enter the housing one-by-one.

4. The instrument of claim 1, wherein the instrument is a reamer and the distal end of the rod includes a cutting edge for cutting the bone and the visual indicator corresponds to a depth of the bone to be cut.

5. The instrument of claim 4, wherein reamer cuts by rotating the rod about a longitudinal axis and the visual indicator is activated by the rotation of the rod.

6. The instrument of claim 1, wherein the visual indicator is one of an LED and a light collecting fiber optic.

7. The instrument of claim 6, wherein the visual indicator is an LED and includes a switch that is activated by the use of the instrument and is de-activated once the LED enters the housing.

8. A method for providing a visual indicator on an instrument for use in implanting a joint prosthesis into a bone, the method comprising:
providing the instrument, the instrument including a housing and a rod, the rod including the visual indicator, the rod being slidably coupled with the housing;
using the instrument such that the visual indicator emits a light;
sliding the rod into the housing until the visual indicator is in the housing; and
causing the light emitted by the visual indicator disappear from view.

9. The method of claim 8, wherein the visual indicator includes a switch and using the instrument comprises actuating the switch.

10. The method of claim 9, wherein the switch is deactivated by the visual indicator entering the housing.

11. The method of claim 8, wherein the visual indicator is a scintillating ambient light collecting fiber optic filament.

12. The method of claim 11, wherein the visual indicator ceases to emit light as the visual indicator enters the housing.

13. The method of claim 11, wherein the scintillating ambient light collecting fiber optic filament is wrapped a plurality of times around the rod.

14. An instrument for use in implanting a joint prosthesis into a bone, the instrument comprising:
a rod having a proximal end and a distal end, the proximal end including a visual indicator; and
a housing, the rod moveable in a longitudinal direction relative to the housing, the visual indicator emitting light when the instrument is in use, wherein as the rod moves in the longitudinal direction, the visual indicator enters into the housing, causing the visual indicator to cease emitting light.

15. The instrument of claim 14, wherein the visual indicator is an LED and includes a switch that is activated when the instrument is in use.

16. The instrument of claim 15, wherein the switch is de-activated when the visual indicator enters the housing.

17. The instrument of claim 14, wherein the visual indicator is a scintillating ambient light collecting fiber optic filament that ceases to emit light when the visual indicator enters the housing.

18. The instrument of claim 14, wherein the visual indicator is a plurality of visual indicators, each of the plurality of visual indicators emitting a different color of light.

19. The instrument of claim 18, wherein each of the plurality of visual indicators corresponds to a measurement of the instrument.

20. The instrument of claim 14, wherein the instrument is a reamer and the distal end of the rod includes a cutting edge for cutting the bone and the visual indicator corresponds to a depth of the bone to be cut.
